(19) United States
(12) Patent Application Publication
(10) Pub. No.: US 2013/0304196 A1
(43) Pub. Date: Nov. 14, 2013

(54) PROSTHETIC VENOUS VALVE HAVING LEAFLETS FORMING A SCALLOPED COMMISSURE

(71) Applicant: John KELLY, Galway (IE)

(72) Inventor: John KELLY, Galway (IE)

(73) Assignee: Medtronic Vascular, Inc., Santa Rosa, CA (US)

(21) Appl. No.: 13/466,268

(22) Filed: May 8, 2012

(51) Int. Cl. A61F 2/06

(52) U.S. Cl. 623/1.25; 623/1.24

(57) ABSTRACT

A prosthetic venous valve includes a self-expanding tubular body defining a fluid passageway and a pair of opposing leaflets biased in a closed configuration in which free edges of the leaflets form a scalloped commissure. The free edges may be pre-formed and/or reinforced in order to ensure sealing with each other in a consistent manner. In response to a pressure differential, the free edges of the leaflets are configured to diverge to form an elliptical outflow opening that allows flow through the tubular body. The valve leaflets may include longitudinal support wires to prevent collapse thereof.

### FIELD OF THE INVENTION

The invention relates to valve prostheses for percutaneous placement within a vein.

### BACKGROUND OF THE INVENTION

Venous valves are self-closing, one-way valves found within native veins and are used to assist in returning blood back to the heart in an antegrade blood flow direction from all parts of the body. The venous system of the leg for example includes the deep venous system and the superficial venous system, both of which are provided with venous valves that are intended to prevent retrograde flow, which can lead to blood pooling or stasis in the leg. Incompetent valves can also lead to reflux of blood from the deep venous system to the superficial venous system and the formation of varicose veins. Superficial veins which include the greater and lesser saphenous veins have perforating branches in the femoral and popliteal regions of the leg that direct blood flow toward the deep venous system and generally have a venous valve located near the junction with the deep venous system. Deep veins of the leg include the anterior and posterior tibial veins, popliteal veins, and femoral veins. Deep veins are surrounded in part by muscular tissues that assist in generating flow by muscle contraction during normal walking or exercising. Blood pressure in the veins of the lower leg of a healthy person may range from 0 mm Hg to over 200 mm Hg, depending on factors such as the activity of the body (i.e., stationary or exercising), the position of the body (i.e., supine or standing), and the location of the vein (i.e., ankle or thigh). For example, venous pressure may be approximately 80-90 mm Hg while standing and may be reduced to 60-70 mm Hg during exercise. Despite exposure to such pressures, the valves of the leg are very flexible and can close with a pressure differential of less than one mm Hg.

FIGS. 1A-1B are schematic representations of the function of a healthy native valve 104 within a vein 100. Valves within the venous system are configured in a variety of shapes that depend on anatomical location, vessel size, and function. For example, the typical shape of the venous valve in man includes two flaps, a.k.a. cusps or leaflets having free edges that sealingly meet, when closed, to form a commissure. Venous valves are typically associated with a broadened area of the vein forming a sinus pocket behind each leaflet. The natural venous valve leaflet configuration referenced herein is for clarity of function and is not limiting in the application of the referenced embodiments. Venous valve 104 controls blood flow through lumen 102 of vein 100 via leaflets 106, 108. More particularly, venous valve 104 opens to allow antegrade flow 112 through leaflets 106, 108 as shown in FIG. 1A. Venous valve 104 closes to prevent backflow or retrograde flow 114 through leaflets 106, 108 as shown in FIG. 1B.

Veins typically in the leg can become distended from prolonged exposure to excessive blood pressure and due to weaknesses found in the vessel wall. Distension of veins can cause the natural valves therein to become incompetent leading to retrograde blood flow in the veins. Such veins no longer function to help pump or direct the blood back to the heart during normal walking or use of the leg muscles. As a result, blood tends to pool in the lower leg and can lead to leg swelling and the formation of deep venous thrombosis and phlebitis. The formation of thrombus in the veins can further impair venous valvular function by causing valvular adherence to the venous wall with possible irreversible loss of venous function. Continued exposure of the venous system to blood pooling and swelling of the surrounding tissue can lead to post phlebitic syndrome with a propensity for open sores, infection, and may lead to limb amputation.

Chronic venous insufficiency (CVI) occurs in patients that have deep and superficial venous valves of their lower extremities (distal to their pelvis) that have failed or become incompetent due to congenital valvular abnormalities and/or pathophysiologic disease of the vasculature. As a result, such patients suffer from varicose veins, swelling and pain of the lower extremities, edema, hyper pigmentation, lipodermatosclerosis, and deep vein thrombosis (DVT). Such patients are at increased risk for development of soft tissue necrosis, ulcerations, pulmonary embolism, stroke, heart attack, and amputations.

FIG. 2 is a schematic representation of retrograde blood flow through an incompetent venous valve. Backflow or retrograde flow 114 leaks through venous valve 104 creating blood build-up that eventually may destroy the venous valve and cause a venous wall bulge 110. More specifically, the wall of vein 100 may expand into a pouch or bulge, such that the vessel has a knotted external appearance when the pouch is filled with blood. The distended vessel wall area may occur on the outflow side of the valve above leaflets 106, 108 as shown in FIG. 2, and/or on the inflow side of the valve below leaflets 106, 108. After a venous valve segment becomes incompetent, the vessel wall dilates and fluid velocity there through decreases, which may lead to flow stasis and thrombus formation in the proximity of the venous valve. Repair and replacement of venous valves presents a formidable challenge due to the low blood flow rate found in native veins, the very thin wall structure of the venous wall and the venous valve, and the ease and frequency of which venous blood flow can be impeded or totally blocked for a period of time. Surgical reconstruction techniques used to address venous valve incompetence include venous valve bypass using a segment of vein with a competent valve, venous transposition to bypass venous blood flow through a neighboring competent valve, and valvuloplasty to repair the valve cusps. These surgical approaches may involve placement of synthetic, allograft and/or xenograft prostheses inside of or around the vein. However, such prostheses have not been devoid of problems, such as thrombus formation and valve failure due to the implanted prostheses causing non-physiologic flow conditions and/or excessive dilation of the vessels with a subsequent decrease in blood flow rates.

Percutaneous transluminal methods for treatment of venous insufficiency are being studied, some of which include placement of synthetic, allograft and/or xenograft prosthesis that suffer from similar problems as the surgically implanted ones discussed above. In light of these limitations, there is a still a need in the art for an improved device that may be percutaneously placed within a vein having an existing insufficient venous valve to re-establish proper flow through the vein segment.

### BRIEF SUMMARY OF THE INVENTION

Embodiments hereof are directed to a prosthetic venous valve including a tubular body defining a fluid passageway and a pair of opposing valve leaflets coupled within the tubular body. In a closed configuration, scallop-shaped free edges of the valve leaflets seal against each other in a commissure that closes the fluid passageway. In an open configuration, the valve leaflet edges diverge away from one another in response to a pressure differential to form an outflow opening having a substantially elliptical shape to allow flow through the fluid passageway.

Embodiments hereof are also directed to a method of controlling blood flow through a vein. A prosthetic venous valve is percutaneously delivered to a treatment site within the vein. The prosthetic valve includes a self-expanding tubular body defining a fluid passageway and a pair of opposing leaflets coupled within the fluid passageway of the tubular body. The prosthetic venous valve is deployed at the treatment site. The valve leaflets of the prosthetic venous valve are biased in a closed configuration in which free edges of the valve leaflets seal in a series of curves or angles at an interface therebetween to prevent blood flow in one direction through the fluid passageway of the tubular body. The free edges of the valve leaflets are configured to diverge in response to a pressure differential to form an outflow opening having a substantially elliptical shape that allows blood flow through the fluid passageway of the tubular body.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIGS. 1A-1B are schematic representations of open and closed configurations in a healthy valve within a vein.

FIG. 2 is a schematic representation of retrograde blood flow through an incompetent valve within a vein.

FIGS. 3-4 are side perspective and end views, respectively, of a prosthetic venous valve having a tubular body and a pair of opposing leaflets coupled to an interior surface of the tubular body, wherein the valve leaflets are in a closed configuration.

FIGS. 5-6 are side perspective and end views, respectively, of the prosthetic venous valve of FIGS. 3-4, wherein the valve leaflets are in an open configuration.

FIG. 7 is a side perspective view of a tubular body of a prosthetic venous valve according to an embodiment hereof.

FIG. 8 is an end view of a prosthetic venous valve in which reinforcing wires are embedded within free edges of the leaflets according to an embodiment hereof.

FIG. 9 is an end view of a prosthetic venous valve having longitudinal support wires on the leaflets according to an embodiment hereof.

FIGS. 10 and 11 are end views of a prosthetic venous valve having alternative commissure configurations according to further embodiments hereof.

FIG. 12 is an example of a delivery system for delivering a prosthetic venous valve.

FIGS. 13 and 14 illustrate a method of percutaneously deploying a prosthetic venous valve.

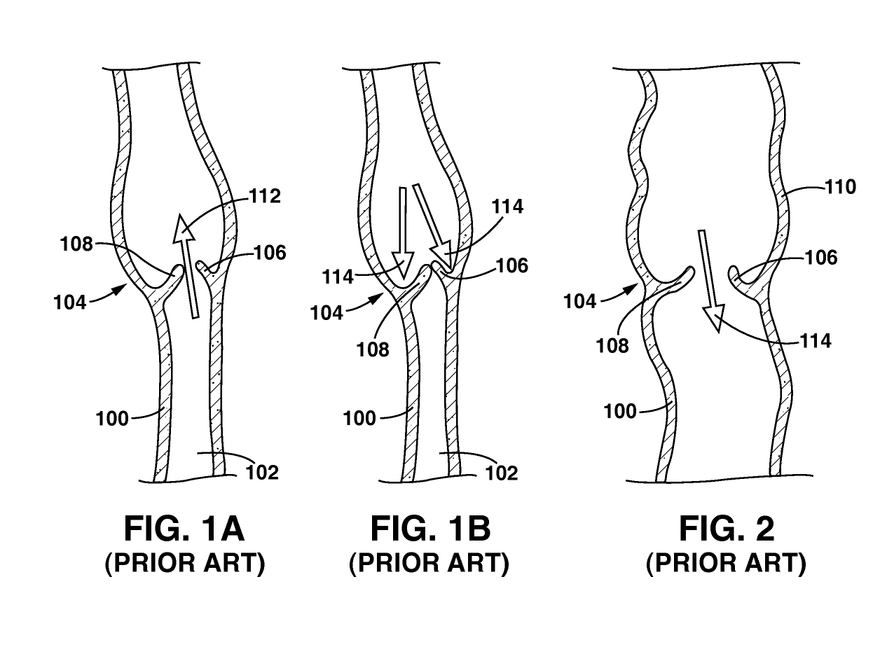

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. In addition, the term “self-expanding” is used in the following description with reference to a tubular body or frame of the valves hereof and is intended to convey that the structures can be provided with a mechanical memory to return the structure from a compressed or constricted delivery configuration to an expanded deployed configuration. Non-exhaustive exemplary self-expanding materials suitable for such structures include stainless steel, a pseudo-elastic metal such as a nickel titanium alloy (nitinol), various polymers, or a nickel-cobalt-chromium-molybdenum superalloy, or other metal. Mechanical memory may be imparted to a wire or tubular structure by thermal treatment to achieve a spring temper in stainless steel, for example, or to set a shape memory in a susceptible metal alloy, such as nitinol. Various polymers that can be made to have shape memory characteristics may also be suitable for use in embodiments hereof, including polymers such as polynorborene, trans-polyisoprene, styrene-butadiene, cross-linked polycyclooctine and polyurethane.

The following detailed description is merely exemplary in nature and is not intended to limit the invention or the application and uses of the invention. Although the description of the invention is in the context of treatment of blood vessels such as veins, the invention may also be used in any other body passageways where it is deemed useful. Furthermore, there is no intention to be bound by any expressed or implied theory presented in the preceding technical field, background, brief summary or the following detailed description.

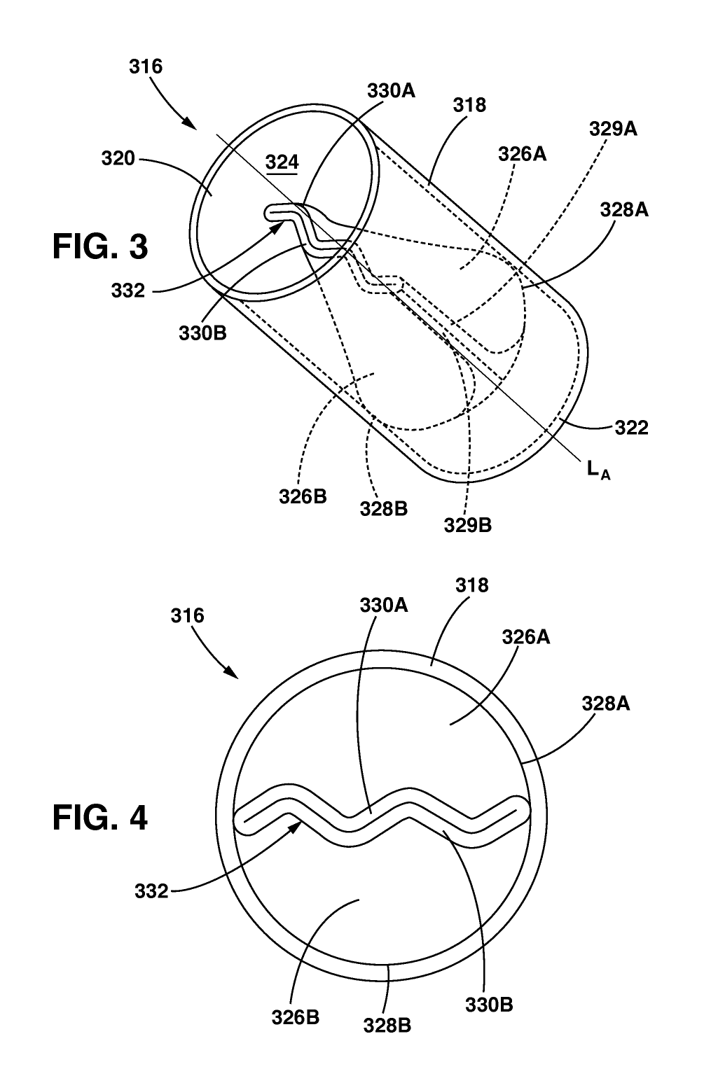

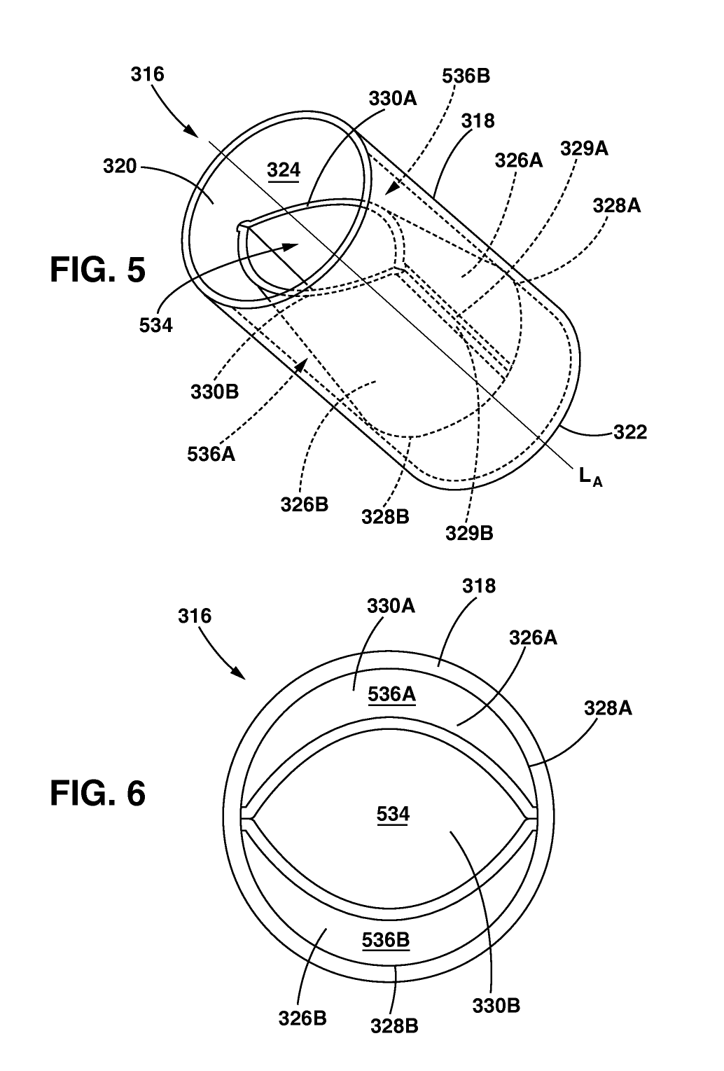

With reference to FIGS. 3-6, an embodiment of a prosthetic venous valve 316 is depicted having a tubular body or conduit 318 defining a lumen or fluid passageway 324 extending from an inlet 322 to an outlet 320 and a pair of opposing leaflets 326A, 326B coupled together and mounted within fluid passageway 324. FIGS. 3 and 4 illustrate a closed configuration of the valve/valve leaflets in which valve leaflets 326A, 326B converge toward one another and seal against each other to prevent retrograde blood flow through fluid passageway 324, i.e. from outlet 320 to inlet 322. FIGS. 5 and 6 illustrate an open configuration of the valve/valve leaflets in which valve leaflets 326A, 326B diverge to allow antegrade blood flow through fluid passageway 324, i.e. from inlet 322 to outlet 320.

In order to mimic the structure of a native venous valve, leaflets 326A, 326B form a substantially elliptical or oval outflow opening 534 when the valve leaflets are in the open configuration shown in FIGS. 5 and 6. As used herein, “substantially elliptical” is intended to mean that outflow opening 534 is not circular but rather in a generally oval or elliptical shape such that the length of a major axis of the opening is greater than the length of a minor axis of the opening. For description purposes, outflow opening 534 is described herein as elliptical but the outflow opening is not required to be an exact geometric ellipse. Elliptical outflow opening 534 mimics the structure of a native venous valve because it creates sinus pockets or sinuses 536A, 536B behind each open leaflet 326A, 326B, respectively. Sinuses 536A, 536B are formed between the outer surface of valve leaflets 326A, 326B and the inner surface of tubular body 318. In addition to providing sinuses that mimic a native venous valve, elliptical outflow opening 534 also mimics the luminal narrowing found in native valves. Lurie et al. reported that, when the venous valve is fully open, the valve cusps create a narrowing of the lumen about 35% smaller than the vein upstream of the valve. Blood flow accelerates through this narrowing, forming a central jet that possibly facilitates outflow. (Lurie F, Kistner R L, Eklof B, Kessler D, Mechanism of venous valve closure and role of the valve in circulation: a new concept, J Vasc Surg. 2003 November; 38(5):955-61) The formation of a central flow jet may also prevent the generation of thrombus in low-flow or static environments. As illustrated in FIG. 6, elliptical outflow opening 534 provides a smaller cross-sectional area than that of fluid passageway 324 through valve body 318. In the open configuration, blood flow enters valve 316 via circular inlet 322, continues through the circular inflow opening created by attached edges 328A, 328B of leaflets 326A, 326B and out elliptical outflow opening 534 created by free leaflet edges 330A, 330B, and exits from valve 316 via outlet 320.

When the elliptical outflow opening 534 closes during operation of prosthetic venous valve 316, there is excess leaflet material because the perimeter of opening 534 is more than twice the diameter of fluid passageway 324. In other words, when free edges 330A, 330B join to form commissure 332, the length of the commissure is greater than the inner diameter of the valve body. Thus, bending or folding of the leaflet material is required in order to form sealing commissure 332 between free leaflet edges 330A, 330B when leaflets 326A, 326B are in the closed configuration. Accordingly, as shown in FIGS. 3 and 4, free edges 330A, 330B of leaflets 326A, 326B form a mating series of sinusoidal curves or zigzag shapes to seal along a commissure 332 in the closed configuration.

For illustrative purposes, valve leaflets 326A, 326B are described herein as independent or separate flaps of material that are coupled to diametrically opposed locations within tubular body 318 and longitudinally extend within tubular body 318. However, in embodiments in accordance herewith the valve leaflets may be integrally formed together as a singular tubular component having a circular inflow opening and an elliptical outflow opening. With reference to FIGS. 3-6, a first or attached end or edge 328A of valve leaflet 326A is coupled to an inside surface of tubular body 318. A first or attached end or edge 328B of valve leaflet 326B is also coupled to an inside surface of tubular body 318 but diametrically opposes attached edge 328A of valve leaflet 326A. Stated another way, opposing leaflets 326A, 326B are mirror images of each other. Longitudinal or side edges 329A, 329B of opposing leaflets 326A, 326B, respectively, are seamed together and may be coupled to an inside surface of tubular body 318. In an alternative embodiment, side edges 329A, 329B may be seemed together but left unattached to tubular body 318, thus leaving first edges 328A, 328B as the only attachments between leaflets 326A, 326B and tubular body 318. Second or free ends or edges 330A, 330B of leaflets 326A, 326B are not attached to the inside surface of tubular body 318, and are configured to meet and abut against each other at interface 332 when the valve leaflets are in a closed configuration.

Valve leaflets 326A, 326B are formed from a biocompatible material such as fabric made from polyethylene terephthalate (PET) fibers also known as polyester and sold under the trademark DACRON. In one embodiment, valve leaflets 326A, 326B may be the same material as a graft material that lines tubular body 318. The inner lining of the tubular body and the valve leaflets may be integrally formed by folding a continuous sheet of the graft material as follows. The inner lining could be allowed to extend past the inlet end 322 of the graft such that this material is then inverted back inside the body to form the leaflets 326A, 326B of the valve. This may involve cutting and stitching of the folded material into the desired shape and position. The lightweight flexible material ensures that valve leaflets 326A, 326B open up or diverge in response to even a low pressure differential across valve 316 and form elliptical outflow opening 534 as shown in FIGS. 5-6. More particularly, valve leaflets 326A, 326B are biased in the closed configuration of FIGS. 3 and 4 with free edges 330A, 330B sealed along sinusoidal commissure 332. The initial shape and configuration of the valve leaflets in addition to the sinusoidal shape set into free edges 330A, 330B will act to bias the valve in the closed position. The pressure differential that occurs during normal blood circulation between the pumped blood on the valve inflow area and the gravity fed blood on the valve outflow area allow valve 316 to operate as a one-way or flow check valve in a similar manner as a natural venous valve. More particularly, when the pumped blood causes the inflow pressure to reach a value greater than the combination of the gravity fed blood pressure and the valve's resistance to opening, valve leaflets 326A, 326B open. The valve's resistance to opening depends on various factors, including the valve leaflet material, the thickness of the valve leaflet material, and the geometry of the valve inflow area. By optimizing these factors, valve 316 is designed to open under inflow pressure conditions that depend on the particular implantation site of the prosthetic valve. As will be described in more detail below, valve 316 is constructed such that valve leaflets 326A, 326B open or diverge to accommodate flow therethrough in response to an actuation pressure PA. In the absence of actuation pressure PA, such as during normal pauses of blood circulation through the body, valve leaflets 326A, 326B resume the closed configuration.

More specifically, when pumped blood is advanced through a vein during normal circulation, blood enters valve 316 through inlet 322 and subjects the interior surface of valve leaflets 326A, 326B to an inlet fluid pressure PI. In venous applications including valves in the lower extremities, PI ranges from 200 mm Hg to 5 mm Hg. When inlet pressure PI equals or exceeds actuation pressure PA, free edges 330A, 330B of leaflets 326A, 326B diverge from one another and form elliptical outflow opening 534 to allow blood flow through valve 316. Generally, venous valve 316 will expand to permit the flow of blood at a rate of about 0.25 L/min to about 5 L/min when the valve leaflets are in the open configuration.

Accordingly, when an actuation pressure PA is reached the venous blood is pumped through valve leaflets 326A, 326B and exits valve 316 through outlet 320. During natural pauses of blood flow, inlet pressure PI is reduced and thus the fluid pressure acting on the interior surface of valve leaflets 326A, 326B decreases. When inlet pressure PI is less than actuation pressure PA, valve leaflets 326A, 326B return to their closed configuration of FIGS. 3-4 in which free edges 330A, 330B seal together to prevent venous blood from backflowing through valve 316. Sinuses 536A, 536B, created by elliptical outflow opening 534 as described above, assist in the closing of valve 316 as backflow fills the sinuses and shifts the pressure differential. Elliptical outflow opening 534 allows for the creation of sinuses 536A, 536B behind respective leaflets 326A, 326B. When valve 316 is open, the pressure in each sinus is less than the pressure in the central channel and this helps keep the valve open. As the flow rate slows, the pressure in the sinus becomes equal to that in the channel and the leaflets begin to close before blood flow has necessarily reversed. Accordingly, once implanted in a vein, venous valve 316 operates as a one-way valve that allows fluid to flow in only an antegrade direction in order to control blood flow through the vein.

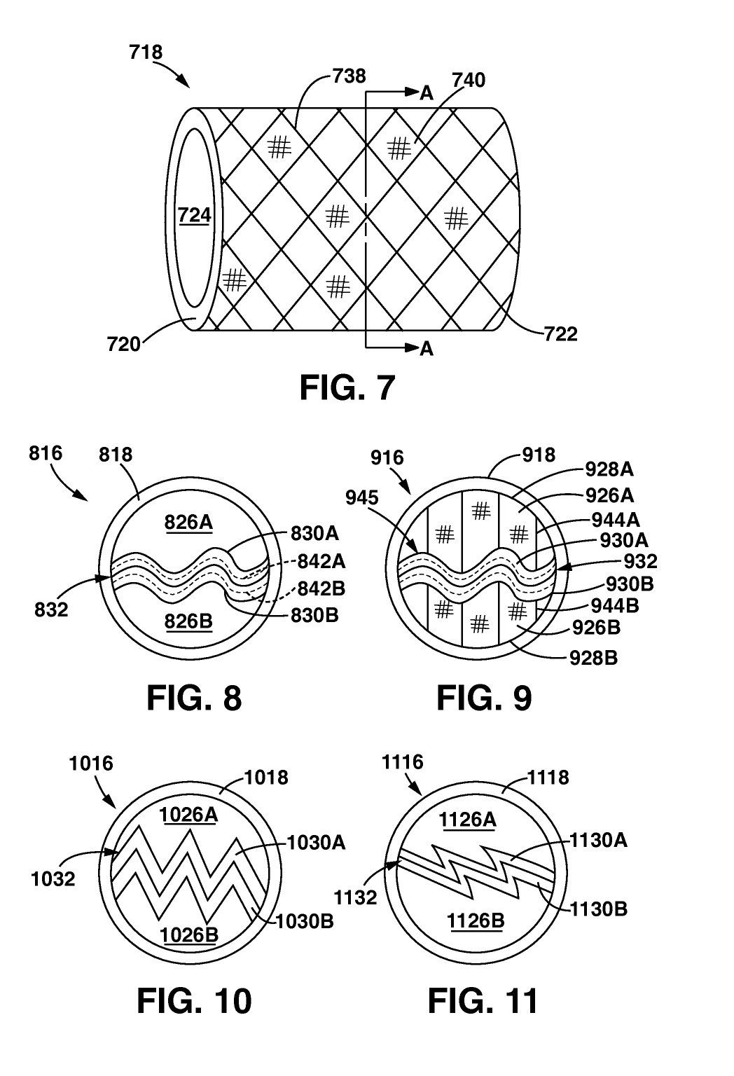

Tubular body 318 of valve 316 is a cylindrical component that defines fluid passageway 324 there through. In one embodiment, the tubular body of the valve is formed from a nitinol reinforced fabric. For example, referring to FIG. 7, tubular body 718 is a radially expandable flexible stent graft constructed from a mesh or lattice scaffolding or stent-like frame 738 having graft material 740 enclosing or lining at least a portion of the stent as would be known to one of ordinary skill in the art. Frame 738 is formed to be self-expanding to return to an expanded deployed configuration from a compressed or constricted delivery configuration, and in an embodiment may be formed from nitinol. Frame 738 allows the valve prosthesis to be compressed and constrained in a radially collapsed state but, when unconstrained, the valve prosthesis will assume an expanded diameter. Further details of such a delivery system and process for deploying self-expanding prostheses as described herein are discussed in further detail below. It will be understood by one of ordinary skill in the art that the valve tubular body may have any configuration that is suitable for forming a fluid passageway through a target vein. As an alternative to being cylindrical, in another embodiment, the frame of the tubular body may have an elliptical or oval cross-section at the midsection thereof. The distal and proximal ends of the frame and the tubular body are circular. The valve, when open, creates an elliptical channel with a sinus behind each leaflet. This narrowed channel effect will act to accelerate blood flow through the valve, which may help prevent thrombosis.

Graft material 740 may be an expanded polytetraflouroethylene (ePTFE) or polyester, which creates a conduit or fluid passageway when attached to frame 738. In one embodiment, graft material 740 may be a knitted or woven polyester fabric. Double or single polyester velour construction can be utilized when it is desired to provide a medium for tissue ingrowth and the ability for the fabric to stretch and conform to a curved surface. These and other appropriate cardiovascular fabrics are commercially available from Bard Peripheral Vascular, Inc. of Tempe, Ariz., for example. In another embodiment, graft material 740 could also be a natural material such as pericardium or another membranous tissue such as intestinal submucosa.

In embodiments hereof, the mating free edges of the valve leaflets may be pre-formed in order to ensure that they bend and seal in a consistent manner when the valve is in a closed configuration. In one embodiment, the mating free edges 330A, 330B of a thermoplastic resin such as polyester are heat set into a desired series of curves or angles to create a sinusoidal or zigzagged interface therebetween when mated in a closed configuration. At least the target edges of the valve leaflets are placed into a die or mold and heat is applied in order to impart a shape memory thereto.

In another embodiment, in addition to or as an alternative to the shape imparted onto the free edges 330A, 330B, a reinforcing element may be coupled to the edges of the leaflets in order to ensure they bend and seal in a consistent manner. For example, referring to FIG. 8, a valve 816 has a tubular body 818 and a pair of valve leaflets 826A, 826B having free edges 830A, 830B, respectively, that meet in a scalloped or sinusoidal commissure 832. A reinforcing wire 842A, 842B (shown in phantom in FIG. 8) is embedded within free edges 830A, 830B of leaflets 826A, 826B, respectively. The wire can be sewn or stitched in place or sandwiched between two layers of material. Each reinforcing wire 842A, 842B has a shape memory of the desired series of curves or angles to ensure that leaflets 826A, 826B bend and seal in a consistent manner. In one embodiment, reinforcing wires 842A, 842B are preformed nitinol wires. Although reinforcing wires 842A, 842B are illustrated as embedded within the edges of leaflets 826A, 826B, it will be understood by those of ordinary skill in the art that the reinforcing wires may be attached to an inside or outside surface of leaflets 826A, 826B.

The valve leaflets may also include longitudinal support wires coupled thereto. For example, FIG. 9 illustrates a valve 916 that has a tubular body 918 and a pair of valve leaflets 926A, 926B having free edges 930A, 930B, respectively, that meet in a scalloped or sinusoidal interface 932. Support wires 944A, 944B are coupled to an outer surface of leaflets 926A, 926B, respectively, to prevent the leaflets from collapsing and unintentionally obstructing the fluid passageway of the valve. In one embodiment, support wires 944A, 944B may extend from free edges 930A, 930B to attached edges 928A, 928B. Further, as shown in FIG. 9, support wires 944A, 944B may extend in parallel to a longitudinal axis L_{A} of the valve (shown in FIG. 3 and FIG. 5) with one end terminating at a peak 945 or valley formed in free edges 930A, 930B. In one embodiment, support wires 944A, 944B are nitinol wires. Although support wires 944A, 944B are illustrated as coupled to the outer surface of leaflets 926A, 926B, it will be understood by one of ordinary skill in the art that the support wires may be attached to an inside surface of leaflets 926A, 926B or may be embedded within the material of leaflets 926A, 926B.

Although embodiments described above illustrate the free edges of the valve leaflets closing at a scalloped or sinusoidal commissure, it will be obvious to one of ordinary skill in the art that other closed configurations are possible. For example, FIG. 10 illustrates a valve 1016 having a tubular body 1018 and a pair of valve leaflets 1026A, 1026B having free edges 1030A, 1030B, respectively, that meet in a zigzagged interface 1032. FIG. 11 illustrates another sealed configuration in which a valve 1116 has a tubular body 1118 and a pair of valve leaflets 1126A, 1126B having free edges 1130A, 1130B, respectively, that meet in a zigzagged or sawtooth-like interface 1132.

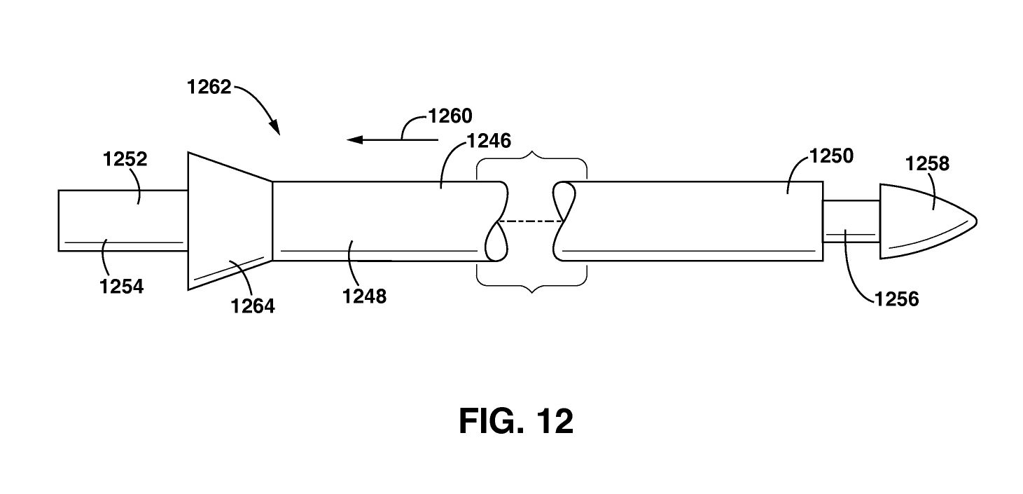

Embodiments of the valve prostheses described herein are preferably delivered in a percutaneous, minimally invasive manner and may be delivered by any suitable delivery system. In contrast to surgically placed valves that require incisions and suturing at the site of a native valve, percutaneous transluminal delivery of a replacement valve can mitigate thromboses formed from an injury response. In general, a venous valve prosthesis having a self-expanding tubular body is loaded into a sheathed delivery system, compressing the self-expanding tubular body. For example, FIG. 12 illustrates a schematic side view of an exemplary delivery system 1262 for delivering and deploying a self-expanding valve prosthesis as described above. The delivery system includes a retractable sheath 1246 having a proximal end 1248 and a distal end 1250, and an inner shaft 1252 having a proximal end 1254 and a distal end 1256 terminating in a distal tip 1258. Distal tip 1258 may be tapered and flexible to provide trackability in narrow and tortuous vessels. Sheath 1246 defines a lumen extending there through (not shown), and inner shaft 1252 slidably extends through the lumen of sheath 1246. In an embodiment, inner shaft 1252 may define a guidewire lumen (not shown) for receiving a guidewire therethrough or may instead be a solid rod without a lumen extending therethrough.

The valve prosthesis (not shown in FIG. 12) may be mounted on distal end 1256 of inner shaft 1252 by any suitable manner known in the art, such as self-expanding attachment bands, a cap coupled to the distal end of the inner shaft to retain the valve prosthesis in a radially compressed configuration, and/or the inclusion of slots, ridges, pockets, or other prosthesis retaining features (not shown) formed into the exterior surface of the inner shaft to secure the valve prosthesis in frictional engagement with the delivery system. Sheath 1246 covers and radially constrains the valve prosthesis while the delivery system is tracked through a body lumen to the deployment site. Sheath 1246 is movable in an axial direction along inner shaft 1252 and extends to a proximal portion of the delivery system where it may be controlled via an actuator, such as a handle 1264, to selectively expand the valve prosthesis. When the actuator is operated, sheath 1246 is retracted over inner shaft 1252 in a proximal direction as indicated by directional arrow 1260. When sheath 1246 is proximally retracted with respect to the hub of the delivery system, the self-expanding valve prosthesis is released and allowed to assume its expanded configuration. An exemplary suitable delivery system is described in U.S. Pat. No. 7,264,632 to Wright et al., which is hereby incorporated by reference in its entirety.

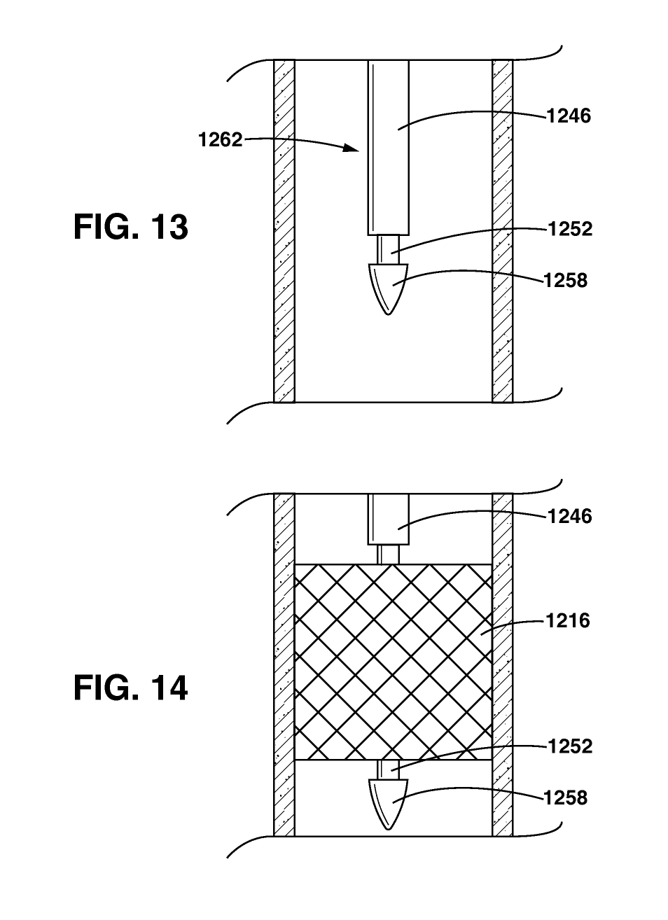

FIGS. 13-14 illustrate a method of controlling flow through a vein by percutaneously delivering a prosthetic venous valve 1216 to a treatment site within the vein. As described in the above embodiments, prosthetic venous valve 1216 includes a self-expanding tubular body defining a fluid passageway and a pair of opposing leaflets coupled to an interior surface of the tubular body. The leaflets of the prosthetic venous valve are biased in a closed configuration in which free edges of the leaflets seal along a sinusoidal or zigzagged commissure to prevent retrograde blood flow through the fluid passageway of the tubular body. Optionally, the prosthetic venous valve may include one or more radiopaque or echogenic markers thereon in order to aid in positioning the valve prosthesis to span across an incompetent native valve.

Referring to FIG. 13, delivery system 1262 is percutaneously introduced into the patient's vasculature. Outer sheath 1246 covers and constrains the valve prosthesis while the delivery system is tracked through a body lumen to the deployment site. Access to the vasculature may be achieved, for example, through the femoral vein. Since the prosthetic valve is a directional one-way valve, it may be loaded into delivery system 1262 in either direction, depending on whether the target site for implantation is to be approached from the antegrade or retrograde direction. Delivery system 1262 is then threaded or tracked through the vascular system of the patient until the prosthetic venous valve is located within a predetermined target site, such as proximate to or over an incompetent native valve within a vein. More particularly, the prosthetic venous valve may be utilized to replace a native incompetent valve or may be deployed between two native valves. When utilized to replace a native incompetent valve, the tubular body of the valve is configured to press radially against the native valve leaflets to hold the native valve leaflets against walls of the native valve annulus and/or against walls of an adjacent lumen to thereby prevent the native valve leaflets from obstructing blood flow through the prosthetic valve. Thus, the prosthetic venous valve may be implanted without requiring removal of a native valve from the vein. In addition, since the tubular body of the prosthetic venous valve spans across the insufficient native valve, the prosthetic venous valve is expected to arrest the progressive damage to the vein caused by the marginal function of the native valve. Blood flow will be directed through the fluid passageway of the prosthetic venous valve and thus bypass any distended or bulged area of the native valve. The damaged venous wall will thus be protected and allowed to scar and/or heal.

Once properly positioned, prosthetic venous valve 1216 is deployed at the treatment site, by retracting sheath 1246 of delivery system 1262 as shown in FIG. 14. Retraction of sheath 1246 allows the tubular body of prosthetic venous valve 1216 to self-expand into apposition with the venous wall, thus securing the prosthetic venous valve within the vein. Once the venous valve prosthesis 1216 is properly deployed at the target site, the delivery system 1262 may be removed from the patient. The free edges of the valve leaflets are configured to diverge in response to a pressure differential to form an elliptical outflow opening that allows flow through the fluid passageway of the tubular body of the prosthetic venous valve.

Although the valve prosthesis is described herein as self-expanding for percutaneous placement, it should be understood that the prosthetic venous valve may alternatively be surgically implanted within a vein in a non-percutaneous manner and may be anchored to the vein in any suitable manner, such as via sutures, clips, or other attachment mechanisms. For example, in such a surgical embodiment, the tubular body of the prosthetic venous valve may include a series of apertures through which sutures can be passed.

Embodiments of the valve prostheses described herein may include an anti-coagulant coating on one or more blood-contacting surfaces of the tubular body and/or the valve leaflets in order to mitigate the formation of thrombus on foreign materials in the bloodstream. In one embodiment, an anti-coagulant material may be embedded in the material of the tubular body and/or the valve leaflets. The anti-coagulant material may be heparin, coumadin, aspirin, ticlopidine, clopidogrel, prasugrel or other suitable anti-coagulant pharmaceuticals. One suitable commercially available product by Carmeda of Sweden offers a clinically proven heparin-based hemocompatible surface coating designed to actively reduce thrombus formation or clotting on blood-contacting medical devices. Carmeda's bioactive surface technology mimics the natural vessel wall to create a blood-compatible surface and also allows for a robust heparin coating to ensure long-term biocompatibility.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A prosthetic venous valve comprising:
a tubular body defining a fluid passageway; and
a pair of opposing valve leaflets, each leaflet having a first edge coupled to an inside surface of the tubular body in a diametrically opposed position to form therebetween a circular inflow opening within the fluid passageway, wherein second free edges of the valve leaflets seal against each other to close the fluid passageway when the valve leaflets are in a closed configuration and diverge away from one another in response to a pressure differential to open the fluid passageway when the valve leaflets are in an open configuration;
wherein when the valve leaflets are in the closed configuration the free edges of the valve leaflets form a series of curves or angles such that the interface therebetween is sinusoidal.

2. The prosthetic venous valve of claim 1, wherein the tubular body includes a self-expanding frame and a lining of graft material for covering at least a portion of the frame.

3. The prosthetic venous valve of claim 1, wherein when the valve leaflets are in the open configuration the free edges of the valve leaflets form an outflow opening having a substantially elliptical shape.

4. The prosthetic venous valve of claim 1, wherein the valve leaflets are biased in the closed configuration with the free edges sealed at the interface therebetween.

5. The prosthetic venous valve of claim 1, wherein at least one of the free edges of the valve leaflets includes a reinforcing wire attached thereto and wherein the reinforcing wire has a shape memory of the series of curves or angles.

6. The prosthetic venous valve of claim 1, wherein the free edges of the valve leaflets are shape set into the series of curves or angles.

7. The prosthetic venous valve of claim 1, wherein at least one of the valve leaflets includes one or more support wires extending between the first and second edges thereof.

8. The prosthetic venous valve of claim 1, wherein the free edges of the valve leaflets form a series of curves in the closed configuration such that the interface therebetween is sinusoidal.

9-19. (canceled)
